(19) United States
(12) Patent Application Publication
(10) Pub. No.: US 2009/0110594 A1
(43) Pub. Date: Apr. 30, 2009

(54) DISH WASHER WITH UV STERILIZATION DEVICE THEREIN

(76) Inventors: Dong Hoon Shin, Gyeongsangnam-do (KR); Soon Yong Kwon, Busan (KR)

Correspondence Address: KED & ASSOCIATES, LLP, P.O. Box 221200, Chantilly, VA, 20153-1200, US

(21) Appl. No.: 11/997,243

(22) PCT Filed: Aug. 17, 2006
(86) PCT No.: PCT/KR2006/003218

§ 371 Date: Dec. 3, 2008

(30) Foreign Application Priority Data
Aug. 19, 2005 (KR) 10-2005-0076144
Oct. 13, 2005 (KR) 10-2005-0096508

(51) Int. Cl. A61L 2/10; A47L 15/00; A47L 15/42

(52) U.S. Cl. 422/24; 134/104.1; 134/57 DL; 134/57 R

(57) ABSTRACT

A dish washer and a control method thereof are disclosed. The dish washer includes a UV sterilization device (60) capable of sterilizing inside of the dish washer and a fan (11) capable of exhausting air in a tub (2) outside. A controller (80) controls the UV sterilization device (60) to sterilize the inside of the dish washer by using ultraviolet rays, and controls the fan (11) to exhaust harmful gas like ozone which may be generated during the sterilization.

### TECHNICAL FIELD

The present invention relates to a dish washer, and more particularly to a dish washer and a control method thereof, which includes a UV sterilization device for sterilizing inside of the dish washer and a fan for exhausting harmful gas which may be generated during the sterilization.

### BACKGROUND ART

Generally, a dish washer is an apparatus for washing dishes contaminated with garbage by spraying water containing detergent, and drying the same.

The dish washer helps a user to conveniently wash the dishes, and is very useful, particularly, for the elderly and the disabled. The dish washer also provides the user with spare time enough to do other works during the operation. So, the demand for the dish washer has been increased recently.

The dish washer is classified into two types. One has a filtering device for collecting the garbage removed from the dishes during the washing. And, the other does not have the filtering device. In the former, the user must eliminate the garbage collected in the filtering device by hand. In the latter, the dish washer has a function of pulverizing the garbage and discharging the same with the water.

FIG. 1 is a side cross-sectional view schematically showing a dish washer of a prior art. As shown in the drawing, a dish washer comprises a main body 1, a tub 2, a driving part 3, washing arms 4, a washing pump 5, and a draining pump 6.

The main body 1 protects the components of the dish washer and forms an exterior appearance. The main body 1 is equipped with a door la at a portion for putting or taking the dishes into or from the tub 2.

The tub 2 is formed inside the main body 1, which provides a space where the dishes are washed. Racks 7 on which the dishes are loaded are provided in the tub 2. Generally, the racks 7 are located at an upper portion and a lower portion in the tub 2.

The driving part 3 for operating the dish washer is mounted under the tub 2. The driving part 3 includes a sump (not shown) for accommodating the water, a heater (not shown) mounted to the sump for heating the water, and a filtering device (not shown) for filtering the garbage removed from the dishes.

The washing arms 4 have a function of spraying the water accommodated in the sump at the dishes loaded on the racks 7. The washing arms 4 are connected to water passages 8 extending from the sump, and respectively mounted under the corresponding racks 7.

The washing pump 5 is mounted under the driving part 3. The washing pump 5 supplies the water accommodated in the sump to the washing arms 4 through the water passages 8.

The draining pump 6 is connected to the filtering device of the driving part 3. The draining pump 6 discharges the water outside, which is used for washing the dishes.

However, since the dish washer of the prior art is not equipped with a device for sterilizing the washed dishes, there is a sanitary problem.

Because the garbage may remain in the dish washer, the dish washer is in danger of being contaminated with bacteria. Especially, the filtering device provides a good environment for the propagation of bacteria.

Further, because the bacteria tend to propagate well in humid environment, the bacteria are not exterminated just by a wet type sterilization device which uses hot water. The bacteria re-propagate due to the humidity of the wet type sterilization device itself.

Accordingly, the dish washer capable of sterilizing the inside of the dish washer, especially, equipped with a dry type sterilization device is required.

### DISCLOSURE OF INVENTION

### Technical Problem

Accordingly, the present invention is directed to a dish washer with a UV sterilization device and a control method thereof that substantially obviate one or more problems due to limitations and disadvantages of the related art.

An object of the present invention devised to solve the problem lies on a dish washer which includes a UV sterilization device capable of sterilizing the inside of the dish washer, especially, a tub or a filtering device.

Another object of the present invention devised to solve the problem lies on a dish washer which includes a fan capable of exhausting the air in the tub outside, thereby removing bad smell from the dish washer and exhausting harmful gas like ozone which may be generated daring the sterilization.

### Technical Solution

The object of the present invention can be achieved by providing a dish washer comprising: a UV emitting device for emitting ultraviolet rays for sterilizing at least a portion of inside of the dish washer; a fan for exhausting air in a tub outside; and a controller for controlling operations of the UV emitting device and the fan so that the fan operates for at least a part of time while the UV emitting device operates.

Preferably, the UV emitting device is a UV lamp or a UV LED. The ultraviolet rays emitted from the UV emitting device exterminate the bacteria. Hereinafter, the UV lamp is a general term including the UV LED.

Preferably, the fan is capable of exhausting air in the tub outside, thereby removing bad smell from the dish washer and exhausting harmful gas like ozone which may be generated during the sterilization.

Preferably, the controller controls the operation of the fan so that the fan operates for predetermined time for completely exhausting the air in the tub from when the operation of the UV emitting device is completed.

Preferably, the UV emitting device emits ultraviolet rays to inside of the tub in which dishes are received. The UV emitting device may be mounted to an inner wall of the tub.

Preferably, the dish washer further comprises: a filtering device for filtering water. The UV emitting device emits the ultraviolet rays to the filtering device. Because the filtering device provides a good environment for the propagation of the bacteria by contaminants like garbage collected therein, the filtering device is necessarily sterilized.

Preferably, the UV emitting device is mounted under the filtering device. This is for preventing the contaminants from building up on the UV emitting device and obstructing the emission of the ultraviolet rays. The contaminants are captured in the filtering device and do not build up on the UV emitting device.

Preferably, the UV emitting device is slantingly mounted, so that the contaminants which may be dropped on the UV emitting device are easily removed from the UV emitting device by being slid down in itself or by the water.

Preferably, the UV emitting device emits the ultraviolet rays to inside of the tub as well as the filtering device. For this, the mounting position of the UV emitting device may be determined properly, or the intensity of the ultraviolet rays may be increased. For example, the UV emitting device may be mounted to the inner wall of the tub or near the filtering device.

When a user takes the dishes from the dish washer before the predetermined sterilization time lapses, it is preferable that the controller controls the UV emitting device to stop and complete the operation. For this, the dish washer further comprises: a detecting device for detecting opening and closing of a door of the dish washer. The controller controls the UV emitting device to operate for predetermined time. When the door is opened and closed before the predetermined time lapses, the controller determines that the dishes have been put out of the tub by a user and controls the UV emitting device to stop and complete the operation. Preferably, a door locking structure or a limit switch may be used as the detecting device.

Preferably, the dish washer further comprises: a heater for heating water for sterilization; and a spraying device for spraying the water heated by the heater. It is preferable to heat the water to a temperature of 80° C.

When the both sterilizations by using hot water and by the UV emitting device are performed, the sterilizing effect is increased by making up for the weak points of both sterilizing methods. In other words, the sterilization using hot water is more effective in sterilizing a driving part having a sump and a washing pump. And, a wave band of the ultraviolet rays emitted from the UV emitting device may be adjusted to effectively exterminate the bacteria which survive the hot water sterilization.

Preferably, the dish washer further comprises: a course selecting button for a user selecting a washing course; and a sterilization selecting button for the user selecting to perform the sterilization using the UV emitting device. Because the sterilization selecting button is provided separately from the course selecting button, only the sterilization can be performed without performing a washing process, thereby providing the user with convenience in use.

Preferably, the dish washer further comprises: a course selecting button for selecting one of multiple washing courses. At least one of the multiple washing courses includes a sterilizing process using a UV emitting device. Therefore, the user can select the washing course having the sterilizing process. The selecting button may be diversely combined, to thereby increase the convenience in use.

In another aspect of the present invention, there is provided a method of controlling a dish washer comprising the steps of: (a) washing dishes received in a tub by using water; (b) rinsing the dishes which are washed; (c) sterilizing the dishes by using water which is heated; and (d) sterilizing at least a portion of inside of the dish washer by using ultraviolet rays.

Preferably, the water is heated to a temperature of 80° C. in the step (C). The step (c) may be performed together with the step (b) at the same time.

Preferably, the method further comprises the steps of: (e) drying the dishes; and (f) exhausting air in the tub outside for at least a part of time while the step (d) is performed.

Preferably, the step (e) is performed after the completion of the step (c), and the step (d) is performed after the completion of the step (e).

Preferably, the step (f) is performed for predetermined time from when the step (d) is completed.

Preferably, when predetermined time lapses from when the step (f) is completed, the step (d) and the step (f) are additionally performed. This is because the user occasionally leaves the dishes in the dish washer even after the completion of the washing procedure from the step (a) to the step (f).

### Advantageous Effects

A dish washer with a UV sterilization device according to the present invention can wash the dishes so they are clean and sanitary, and maintain the inside of the dish washer in a hygienic state by sterilizing the inside (especially, a tub or a filtering device) of the dish washer.

The dish washer according to the present invention includes a fan capable of exhausting air in the tub outside, thereby removing bad smell from the dish washer and exhausting harmful gas like ozone which may be generated during the sterilization.

Also, when the sterilization using hot water and the UV sterilization are performed at the same time, the sterilizing effect is increased by making up for the weak points of both sterilizing methods.

### BRIEF DESCRIPTION OF THE DRAWINGS

The accompanying drawings, which are included to provide a further understanding of the invention, illustrate embodiments of the invention and together with the description serve to explain the principle of the invention.

In the drawings:

FIG. 1 is a side cross-sectional view schematically showing a dish washer of a prior art;

FIG. 2 is a block diagram showing a constitution of a dish washer with a UV sterilization device in accordance with the present invention;

FIG. 3 is a side cross-sectional view schematically showing a dish washer with a UV sterilization device in accordance with a preferred embodiment of the present invention;

FIG. 4 is a cross-sectional view showing a dish washer with a UV sterilization device in accordance with another preferred embodiment of the present invention;

FIG. 5 is a schematic view showing an example of a control panel of a key input part;

FIG. 6 is a schematic view showing another example of a control panel of a key input part;

FIG. 7 is a flow chart showing a method of controlling a dish washer in accordance with the present invention; and

FIG. 8 is a flow chart showing an operation of a dish washer in accordance with the present invention.

### BEST MODE FOR CARRYING OUT THE INVENTION

Reference will now be made in detail to the preferred embodiments of the present invention, examples of which are illustrated in the accompanying drawings.

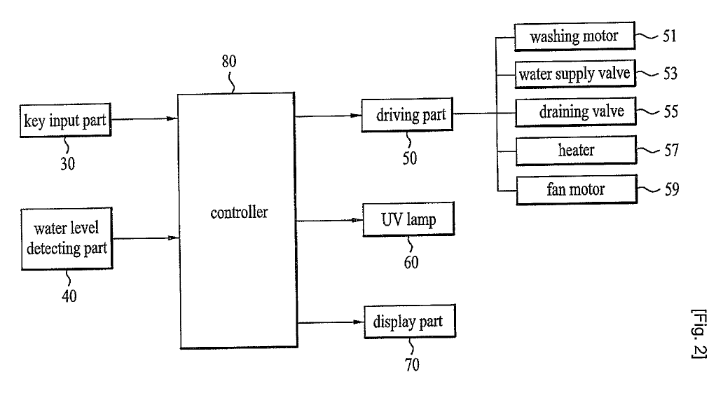

FIG. 2 is a block diagram showing a constitution of a dish washer with a UV sterilization device in accordance with the present invention.

As shown in the drawing, a dish washer of the present invention comprises a key input part 30 for selecting a washing course and whether to operate a sterilization function or not; a water level detecting part 40 for detecting the water level in a tub; a driving part 50 for operating a washing motor 51, a water supply valve 53, a draining valve 55, a heater 57 and a fan motor 59; a UV lamp (or a UV LED) 60 for emitting ultraviolet rays for sterilizing the dishes which are completely washed and dried; a display part 70 for displaying a washing procedure; a controller 80 for controlling a load necessary for the washing procedure and controlling the UV lamp 60 for sterilizing the dishes and the driving part 50 to operate the fan motor 59 for ventilating the tub when washing, rinsing and drying processes are finished.

When the drying process is finished, the UV lamp 60 emits the ultraviolet rays to sterilize the dishes for a predetermined time. In addition, during the operation of the UV lamp 60, the fan motor 59 is driven to transmit its driving force to a fan for exhausting air in the tub outside.

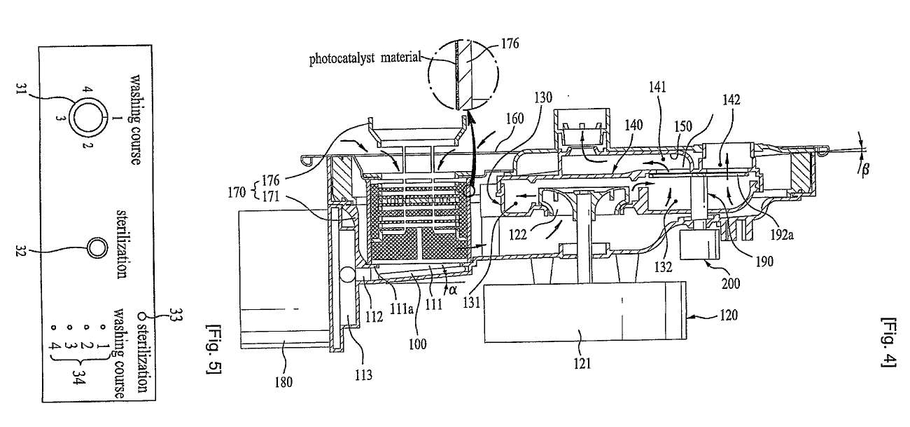

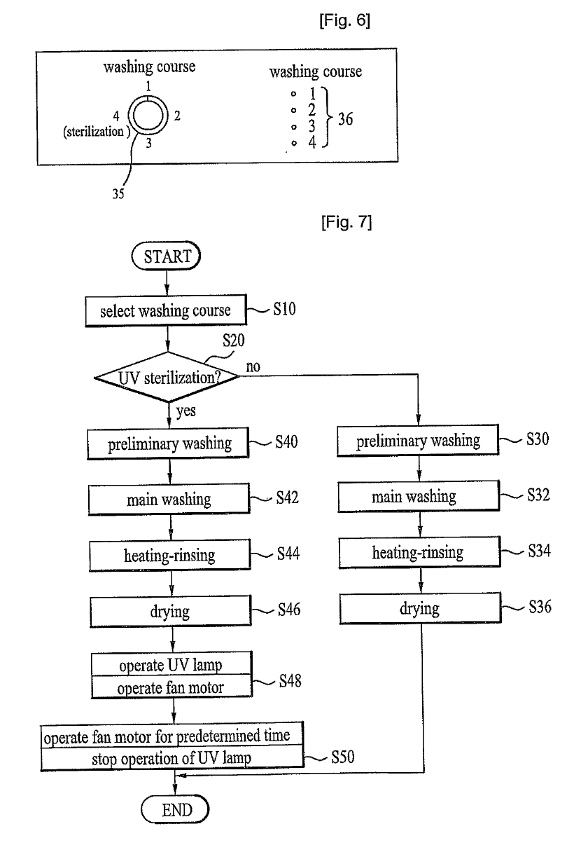

The key input part 30 may include control panels which are shown FIGS. 5 and 6. The control panel depicted in FIG. 5 is provided with a washing course selecting knob 31 for selecting the washing course and a sterilization selecting button 32 for allowing a user to select whether to perform the UV sterilization or not. The commands selected by manipulating the washing course selecting knob 31 and the sterilization selecting button 32 are displayed through LEDs (Light Emitting Diodes) 33 and 34, to thereby inform the user of the selected commands.

The control panel depicted in FIG. 6 is provided with a washing course selecting knob 35 for selecting the washing course. One of the programmed washing courses includes a UV sterilizing process. In other words, if the user selects a “washing course 4” (see FIG. 6), the UV sterilizing process is performed while the “washing course 4” is performed. The command selected by manipulating the washing course selecting knob 35 is displayed through LEDs 36.

Hereinafter, a method of controlling the dish washer according to the present invention will be described with reference to FIG. 7.

The user loads the contaminated dishes on racks in the tub. And, the user selects the washing course and whether to perform the sterilization or not in consideration of the amount and kinds of the dishes to be washed by manipulating the key input part 30 at step S10.

The controller 80 determines if the washing course including the sterilizing process is selected by the user at step S20. If the controller 80 determines that the washing course selected by the user does not include the sterilizing process, the controller 80 performs the washing procedure according to algorithm corresponding to the selected washing course without performing the sterilizing process.

First, the water supply valve 53 is opened to supply the water, and the water level detecting part 40 detects the water level. When the water level detected by the detecting part 40 reaches a predetermined value, the water supply valve 53 is closed to stop the water-supply. By repeatedly performing and stopping the spraying of the water containing the detergent at the dishes loaded on the racks in the tub at predetermined times, a preliminary washing process is performed at step S30.

When the preliminary washing process is completed, the draining valve 55 is opened to discharge the water used in the preliminary washing process, and the water supply valve 53 is opened to supply again the water to the predetermined water level. By driving the heater 57 to heat the water and spraying the heated water at the dishes, a main washing process is performed at step S32.

When the main washing process is completed, the draining valve 55 is opened to discharge the water used in the main washing process, and the water supply valve 53 is opened to again supply the water. By heating the water and using the heated water, a heating-rinsing process is performed to remove the remaining garbage or the detergent from the dishes at step S34.

At this time, it is preferable to heat the water to a temperature (e.g., about 80° C.) enough for the sterilization. Therefore, the rinsing and the sterilization using the hot water are performed at the same time.

When the preliminary washing process, the main washing process and the heating-rinsing process are completed, by driving the fan motor 59 to rotate the fan for exhausting the air in the tub outside and removing the water from the dishes, a drying process is performed at step S36.

Conclusively, in case of the washing course which does not include the sterilizing process, the preliminary washing process, the main washing process, the heating-rinsing process and the drying process are performed in order.

On the other hand, if the user selects the washing course including the sterilizing process, by repeatedly performing and stopping the spraying of the water containing the detergent at the dishes, a preliminary washing process is performed at step S40. When the preliminary washing process is completed, by driving the heater 57 to heat the water and spraying the heated water at the dishes, a main washing process is performed at step S42. When the main washing process is completed by heating the water and using the heated water, a heating-rinsing process is performed at step S44. When the heating-rinsing process is completed, by driving the fan motor 59 to rotate the fan for exhausting the air in the tub outside and removing the water from the dishes, a drying process is performed at step S46.

When the ding process is completed, in order to sterilize the dried dishes and remove the garbage smell from the dishes, the UV lamp 60 and the fan motor 59 are driven for predetermined time (e.g., for 25 minutes or more) at step S48. In other words, the UV lamp 60 is driven to sterilize the dishes, and the fan motor 59 is driven to rotate the fan for exhausting the air and the harmful gas like ozone in the tub, which may be generated during the sterilizing process, and removing the bad smell.

When the predetermined time for driving the UV lamp 60 passes, the operation of the UV lamp 60 is stopped, but the fan motor 59 is continuously driven to sufficiently ventilate the tub for predetermined time (e.g., for about 35 minutes) at step S50.

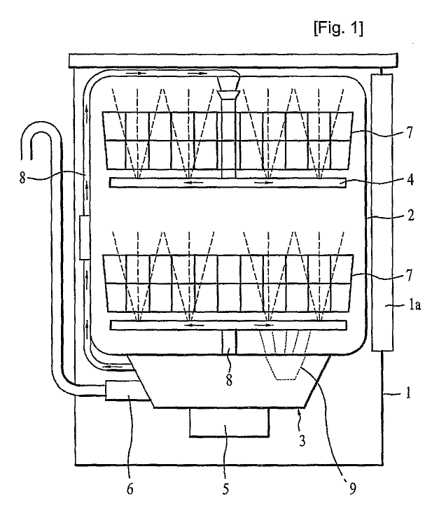

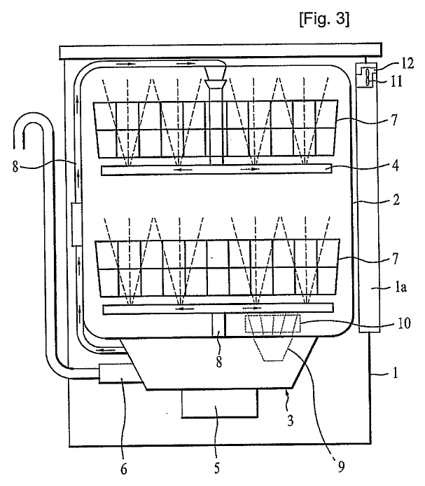

FIG. 3 is a side cross-sectional view schematically showing the dish washer in accordance with a preferred embodiment of the present invention. Same reference numerals are given to the same components of the dish washer of the present invention as the components of the dish washer of the prior art depicted in FIG. 1, and detailed explanation thereof will be omitted.

As shown in FIG. 3, the dish washer comprises a UV lamp 10 provided near a filtering device 9 in a tub 2, a fan 11 mounted to a door 1a for exhausting the air in the tub 2 outside, and a controller (not shown) for controlling the UV sterilizing process.

It is illustrated in FIG. 3 that the UV lamp 10 is located in the tub 2 near the filtering device 9, however, the position of the UV lamp 10 may be diversely changed. The filtering device 9 filters the contaminants like the garbage out of the water which is sprayed from washing arms 4 and used for washing the dishes. The ultraviolet rays emitted from the UV lamp 10 can reach the filtering device 9 as well as the inside of the tub 2, thereby sterilizing them together.

The door 1a is formed with an air passage 12, and the fan 11 is located in the air passage 12. The fan 11 rotates by a driving force of a fan motor. As the fan 11 rotates, the air in the tub 2 is exhausted outside.

The controller controls the operation of the UV lamp 10 to sterilize the dishes, and controls the operation of the fan 11 to exhaust the bad smell and the harmful gas in the tub 2 outside. Because the controller can control the operation of the UV lamp 10 for the UV sterilization according to the control method described above with reference to FIG. 7, the explanation of the control method will be omitted.

FIG. 4 is a cross-sectional view showing a dish washer in accordance with another preferred embodiment of the present invention. FIG. 4 illustrates only a driving part which is mounted under the tub. The overall structure except the driving part of this embodiment may be identical to the structure of the previous embodiment. On the other hand, the dish washer of this embodiment may not include the UV lamp 10 of the previous embodiment (see FIG. 3).

As shown in FIG. 4, the dish washer comprises a sump 110 for accommodating the water, a washing pump 120 for pumping the water in the sump 110, housing assembly 130, 140 and 150 having water passages for guiding the pumped water to washing arms, a cover 160 for covering an upper portion of the sump 110, a filtering device 170 mounted in the sump 110 while passing through the cover 160 for filtering the water dropped on the cover 160 and guiding the filtered water into the sump 110, a draining pump 180 communicated with the filtering device 170 for discharging the water in the sump 110 outside, a UV lamp 100 mounted near the lower end of the filtering device 170 for sterilizing the filtering device 170, and a heater (not shown) mounted in the sump 110 for heating the water.

The sump 110 is formed with a contaminant accommodating chamber 111. It is preferable that the contaminant accommodating chamber 111 is disposed at the bottom of sump 110. It is further preferable that the contaminant accommodating chamber 111 is slanted toward the draining pump 180 by an angle of, so as to easily exhaust the contaminants collected in the contaminant accommodating chamber 111 to the draining pump 180.

A discharging passage 112 is formed between the contaminant accommodating chamber 111 and the draining pump 180 to communicate them with each other. The sump 110 is formed with a draining chamber 113 for mounting the draining pump 180. It is preferable that the contaminant accommodating chamber 111 communicates with the draining chamber 113 through the discharging passage 112. The draining chamber 113 is provided with an impeller (not shown).

The washing pump 120 includes a washing motor 121 for generating a driving force, and an impeller 122 which is shaft-coupled to the washing motor 121. The washing motor 121 is disposed such that its driving shaft extends vertically. The draining pump 180 includes a draining motor (not shown) and an impeller (not shown). The draining motor is disposed such that its driving shaft extends horizontally.

The housing assembly 130, 140 and 150 includes a pump chamber 131 in which the impeller 122 of the washing pump 120 is located, a flow control chamber 132 which communicates with the pump chamber 131 and in which a flow control valve 190 is mounted, and main water passages 141 and 142 for guiding the water in the flow control chamber 132 to the washing arms.

It is preferable to dispose the pump chamber 131 and the flow control chamber 132 on the same plane. This is for decreasing a height of the housing assembly 130, 140 and 150 and increasing the inner space of the sump 110. It is preferable to dispose the main water passages 141 and 142 on the pump chamber 131 and the flow control chamber 132. This is for decreasing an area of the housing assembly 130, 140 and 150.

Here, the housing assembly will now be described in detail. The housing assembly includes a lower housing 130 which has the pump chamber 131 and the flow control chamber 132, and an upper housing 140 which covers an upper portion of the lower housing 130 and has the main water passages 141 and 142. The lower housing 130 and the upper housing 140 may be formed as separate members as shown in FIG. 4, or may be formed integrally with each other.

The housing assembly may further include a connecting housing 150 which covers an upper portion of the upper housing 140. A connecting pipe (not shown) connected to the washing arms is coupled to the connecting housing 150. The connecting housing 150 is formed with a coupling part (not shown) to which the connecting pipe is coupled.

It is preferable that the cover 160 which covers the sump 110 is slanted toward the filtering device 170 by an angle of β. This is in order that the water and the contaminants dropped on the cover 160 can easily flow to the filtering device 170.

It is preferable to form a plurality of filtering holes 161 at the cover 160 for allowing a part of the filtered water to directly flow into the tub. It is more preferable to dispose the filtering holes 161 at the positions with the exception of the housing assembly 130, 140 and 150. This is for minimizing the contamination of the outer surface of the housing assembly 130, 140 and 150 by the dirty water.

The filtering device 170 has an opened lower end. The opened lower end of the filtering device 170 is coupled to the contaminant accommodating chamber 111. It is preferable that the lower end of the filtering device 170 is spaced apart from a bottom of the contaminant accommodating chamber 111.

A stepped portion is formed at an upper end of the contaminant accommodating chamber 111, so that the opened lower end of the filtering device 170 is supported by the stepped portion. The discharging passage 112 is disposed under the stepped portion of the contaminant accommodating chamber 111. Therefore, the contaminants in the contaminant accommodating chamber 111 are exhausted to the draining chamber through the discharging passage 112 without interference with the filtering device 170.

The filtering device 170 includes an upper filter 176 and a lower filter 171. The upper filter 176 and the lower filter 171 are detachably coupled to the cover 160. The upper filter 176 filters relatively large garbage, and the lower filter 171 filters relatively small garbage which is not filtered by the upper filter 176. Preferably, the lower filter 171 is coated with a photocatalyst material.

The upper filter 176 may protrude from the upper surface of the cover 160 by a predetermined height, so that the user can easily grasp the upper filter 176 and draw out the same. On the other hand, if the upper filter 176 has a structure that the user can easily draw out, the upper filter 176 does not necessarily protrude from the upper surface of the cover 160.

The UV lamp 100 is disposed near the lower end of the lower filter 171. The sterilizing effect varies according the position to which the UV lamp 100 emits the ultraviolet rays. It is most effective when the UV lamp 100 is located near the lower end of the lower filter 171 and emits the ultraviolet rays to the filtering device 170. Accordingly, it is preferable to dispose the UV lamp 100 at a side of the contaminant accommodating chamber 111. And, the UV lamp 100 is slantingly mounted to prevent the contaminants from building up thereon.

The flow control valve 190 includes a rotating shaft 191 which is coupled to a driving shaft of a control motor 200, and an opening/closing plate 192 which is mounted at a top of the rotating shaft 191 and is formed with a communicating hole 192a. As the opening/closing plate 192 rotates, the plate 192 opens or closes the main water passages 141 and 142. Because the main water passages 141 and 142 are located on the flow control valve 190, the flow control valve 190 can be formed in a plate shape. Also, since the pumping pressure of the washing pump 120 acts on the upper portion of the flow control valve 190, the pumping pressure biases the flow control valve 190 to the upper housing 140, so that the flow control valve 190 can be stably supported without rattling.

Preferably, the inner surfaces of the sump 110 and the main water passages 141 and 142 are coated with a nano-silver material. This is because the nano-silver material having an antifungal effect prevents the bacteria from propagating due to the water remaining in the sump 110 and the main water passages 141 and 142.

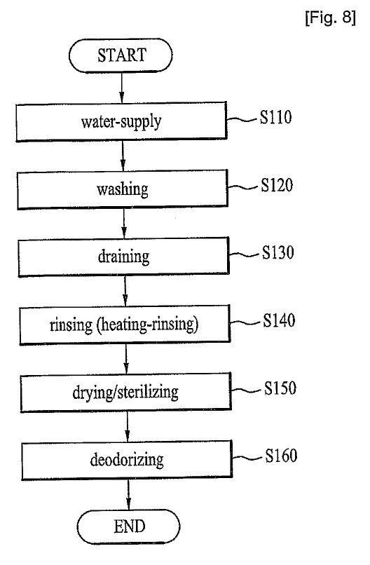

Hereinafter, the operation of the dish washer structured as above will be described with reference to FIG. 8. The dish washer operates by a water supplying process, a washing process, a draining process, a heating-rinsing process, a drying/sterilizing process, and a deodorizing process.

The water supplying process is performed at step S110 in such a manner that the water is supplied into the sump 110 with the detergent. The water is supplied to the predetermined level.

Then, the washing process is performed at step S120. When the washing motor 121 is driven, the impeller 122 rotates and the water in the sump 110 flows to the pump chamber 131 and the flow control chamber 132. At this time, the flow control valve 190 rotates to communicate the main water passages 141 and 142 with the flow control chamber 132. The flow control valve 190 may selectively open one of the main water passages 141 and 142, or may simultaneously open both the main water passages 141 and 142. Also, the flow control valve 190 may alternately open and close the main water passages 141 and 142.

When the main water passages 141 and 142 are opened, the water in the flow control chamber 132 flows to the washing arms through the main water passages 141 and 142, and is sprayed from the washing arms. Therefore, since the amount of the water sprayed at the dishes is increased and substantially all the pumped water is used only for washing the dishes, the washing pump 120 having relatively smaller capacity can be used, and the consumption of the water is considerably decreased.

After being sprayed from the washing arms and used for washing the dishes, the water drops on the cover 160. Because the cover 160 is slanted toward the filtering device 170, the water and the contaminants dropped on the cover 160 flow to the upper filter 176. A part of the water directly flows into the sump 110 through the filtering holes 161 of the cover 160.

The upper filter 176 filters the relatively large garbage, and the lower filter 171 filters the relatively small garbage which is not filtered by the upper filter 176. As a result, only the water from which the contaminants are filtered flows into the sump 110.

When the washing process is performed for the predetermined time, the dishes are washed while the contaminants are filtered from the water. As the washing time passes, the amount of the contaminants like the garbage collected in the filtering device 170 is gradually increased.

When the washing process is completed, the draining process is performed at step S130. When the draining pump 180 is driven, the water in the sump 110 flows into the contaminant accommodating chamber 111 through the lower filter 171 by a sucking force of the draining pump 180. Then, the water flows into the draining chamber 113 with the contaminants collected in the contaminant accommodating chamber 111. Because the contaminant accommodating chamber 111 is slanted toward the draining chamber 113, the contaminants can smoothly flow into the draining chamber 113. The water and the contaminants in the draining chamber 113 are discharged outside through a draining hole and a draining hose of the draining chamber 113.

When the draining process is completed, the rinsing (preferably, the heating-rinsing) process is performed at step S140. It is preferable that the rinsing and the sterilization using the hot water are performed simultaneously. The water is heated by the heater mounted in the sump 110 to the predetermined temperature, and the heated water is sprayed to the dishes from the washing arms, thereby increasing the rinsing ability and sterilizing the dishes at the same time. Preferably, the water is heated to the temperature of about 80° C.

When the heating-rinsing process is completed, the water is discharged. Then, the drying/sterilizing process is performed at step S150. Here, the sterilization means a UV sterilization.

The UV lamp 100 emits the ultraviolet rays to the filtering device 170, to sterilize the filtering device 170 and the contaminant accommodating chamber 111. Since the surface of the lower filter 176 is coated with the photocatalyst material, the photocatalyst material receives the ultraviolet rays emitted from the UV lamp 100 and generates the chemical reaction, to reinforce the sterilizing effect. Accordingly, the lower filter 176 in which the minute contaminants may remain can be sterilized more intensively.

While the sterilizing process is performed and after completion, the deodorizing process is performed at step S160. The fan mounted to the door rotates to exhaust the harmful gas like ozone which may be generated during the operation of the UV lamp. Here, the deodorizing process means a ventilating process.

It will be apparent to those skilled in the art that various modifications and variations can be made in the present invention without departing from the spirit or scope of the invention. Thus, it is intended that the present invention covers the modifications and variations of this invention provided they come within the scope of the appended claims and their equivalents.

### INDUSTRIAL APPLICABILITY

The dish washer according to the present invention has structural features of including the UV sterilizing device capable of sterilizing the inside of the dish washer and the fan capable of exhausting the harmful gas which may be generated during the sterilizing process. The fan also exhausts the air in the tub outside, so the bad smell in the dish washer is removed.

Because the inside (especially, the tub or the filtering device) of the dish washer can be sterilized, the dishes can be washed so they are clean and sanitary clean, and the inside of the dish washer can be maintained in a hygienic state.

Further, when the sterilization using the hot water and the UV sterilization are performed at the same time, the sterilizing effect is increased by making up for the weak points of both sterilizing methods.

Of course, the present invention can be similarly applied to the different types of the dish washers which do or do not have the filtering device.

1. A dish washer comprising:
a UV emitting device for emitting ultraviolet rays for sterilizing at least a portion of inside of the dish washer;
a fan for exhausting air in a tub outside; and
a controller for controlling operations of the UV emitting device and the fan so that the fan operates for at least a part of time while the UV emitting device operates.

2. The dish washer according to claim 1, wherein the controller controls the operation of the fan so that the fan operates for predetermined time from when the operation of the UV emitting device is completed.

3. The dish washer according to claim 1, wherein the UV emitting device emits ultraviolet rays to inside of the tub in which dishes are received.

4. The dish washer according to claim 3, further comprising:
a detecting device for detecting opening and closing of a door of the dish washer,
wherein the controller controls the UV emitting device to operate for predetermined time, and
when the controller determines that the door is opened and closed before the predetermined time lapses, the controller controls the UV emitting device to stop and complete the operation.

5. The dish washer according to claim 1, further comprising:
a heater for heating water for sterilization; and
a spraying device for spraying the water heated by the heater.

6. The dish washer according to claim 1, further comprising:
a filtering device for filtering water,
wherein the UV emitting device emits the ultraviolet rays to the filtering device.

7. The dish washer according to claim 6, wherein the UV emitting device emits the ultraviolet rays also to inside of the tub.

8. The dish washer according to claim 6, wherein the UV emitting device is mounted under the filtering device.

9. The dish washer according to claim 8, wherein the UV emitting device is slantingly mounted.

10. The dish washer according to claim 1, further comprising:
a course selecting button for selecting a washing course; and
a sterilization selecting button for performing sterilization using the UV emitting device.

11. The dish washer according to claim 1, further comprising:
a course selecting button for selecting one of multiple washing courses,
wherein at least one of the multiple washing courses includes a sterilizing process using the UV emitting device.

12. A method of controlling a dish washer comprising the steps of:
washing dishes received in a tub by using water;
rinsing the washed dishes;
sterilizing the dishes by using heated water; and
sterilizing at least a portion of inside of the dish washer by using ultraviolet rays.

13. The method according to claim 12, further comprising the steps of:
drying the dishes; and
exhausting air in the tub outside for at least a part of time while the step of sterilizing by using ultraviolet rays is performed.

14. The method according to claim 13, wherein the step of exhausting air is further performed for predetermined time after the step of sterilizing by using ultraviolet rays is completed.

15. The method according to claim 14, wherein after predetermined time lapses from when the step of exhausting air is completed, the step of sterilizing by using ultraviolet rays and the step of exhausting air are additionally performed.

16. A method of controlling a dish washer comprising the steps of:
washing dishes received in a tub by using water;
rinsing the dishes by using water which is heated for sterilization;
drying the dishes which are rinsed;
sterilizing the dishes by using ultraviolet rays; and
exhausting air in the tub outside while the step of sterilizing by using ultraviolet rays is performed and after the step of sterilizing by using ultraviolet rays is completed.
